(19) United States
(12) Patent Application Publication
(10) Pub. No.: US 2023/0279233 A1
(43) Pub. Date: Sep. 7, 2023

(54) ATROROSINS AS FOOD COLORS

(71) Applicant: Chromologics ApS, Søborg (DK)

(72) Inventors: Henrik Dalbøge, Virum (DK); Anders Sebastian Rosenkrans Ødum, Nærum (DK)

(21) Appl. No.: 18/000,432

(22) PCT Filed: Jul. 6, 2021
(86) PCT No.: PCT/EP2021/068647

§ 371 Date: Dec. 1, 2022

(30) Foreign Application Priority Data
Jul. 6, 2020 (DK) PA202070464

(51) Int. Cl. C09B 61/00; A23L 5/46; B01D 61/14; A23L 2/58; A23G 3/36; C09B 63/00; A61K 8/49; A61Q 1/06; C12P 17/18; C12N 1/14

(52) U.S. Cl. CPC C09B 61/00; A23L 5/46; B01D 61/145; A23L 2/58; A23G 3/36; C09B 63/005; A61K 8/4926; A61Q 1/06; C12P 17/188; C12N 1/14; A61K 2800/43

(57) ABSTRACT

The present invention provide scalable methods of producing newly identified atrorosin pigments, compositions comprising the pigments, lakes and dyes and uses thereof for colorings foods, such as dairy, meat substitutes or candy.

### FIELD OF INVENTION

The present invention relates to using newly identified pigments, atrorosins, from Talaromyces atroroseus for colorings foods, such as dairy, meat substitutes or candy.

The present invention further relates to a method of preparing improved atrorosin food coloring compositions and the uses of such a compositions.

### BACKGROUND

Colorants in food are either of synthetic or natural origin, and can be dyes or pigment, depending on how/if the color is integrated in the given matrix. Dyes are soluble in the matrix and color by suspension, whereas pigments are insoluble in the matrix and color by dispersion. In general, in the food industry, dyes are water soluble and pigments are insoluble in water.

Food colorants can be categorized as natural, nature-identical, or synthetic. Natural colorants are pigments or dyes found in nature, biosynthesized by a living organism. They are mainly plant extracts but can also be extracted from insects¹. Nature-identical colorants are colors that are chemically synthesized or semi-synthesized pigments with identical chemical structures to those found in nature, such as beta-carotene. Synthetic colors are purely chemically synthesized colors typically organic compounds containing an azo coupling and based on petroleum².

During the last decade, consumer chemistry has become a focal point of interest. This includes fragrances, flavors, and colorants in food and non-food products. In foods, most companies have already switched from synthetic to natural colorants as they are perceived to be more healthy, safe, and authentic.^{2,3} There is a strong market pull for high performing natural colorants in industry segments such as food, cosmetics, and home care. Red is the colorant with the broadest application range within foods and takes up to 55% of the market share. Industrially used natural red colorants are primarily extracted directly from natural sources e.g. betanin (beetroot, Beta vulgaris, extract), lycopene (tomato Solanum lycopersicum extract) or carminic acid (extracted from females of the insect Dactylopius coccus). These have different limitations in performance regarding pH and temperature stability, and solubility, or sourcing issues such as carminic acid deriving from insects making them unsuitable for kosher, halal, or vegetarian/vegan diets. The dependence on specific raw materials can cause problems in the cost structure. This includes volatile pricing caused by seasonal variations and changes in quantity/quality of the harvests (e.g. insects on cacti), and high pricing due to price intensive extraction methods (lycopene form tomatoes)⁴.

Betanin and Carmine are the two most abundant used red natural food colorants. Betanin is extracted from beetroot. There are two drawbacks associated with betanin: a) very poor temperature stability and b) a characteristic off taste. Temperature stability is critical for many manufacturing processes and lack of stability over 40° C. is a major problem for betanin^{5,6}. Food manufacturing companies try to circumvent the lack of stability by oversaturation of the colorant⁷. This however increases the unwanted off taste. Betanin's lack of temperature stability makes it unsuitable for its application in meat substitutes since heat treatment is involved either in the manufacturing process or later by the end consumer.

Carminic acid is extracted from the insects Dactylopius coccus⁸. It has good temperature stability retaining a high color concentration even after being heated at 90° C. for 20 minutes. One drawback of carminic acid is its pH sensitivity. This means carminic acid change its color depending on the pH of the matrix. It is orange/yellow below pH 4, red from pH 4-6 and above pH 6 purple/red. Carminic acid can be made into a lake pigment by coupling carminic acid to aluminum making it insoluble in water. Carmine lake however is only stable in alkaline solutions above pH 6. The biggest issue with carminic acid/carmine however, is its extraction source (insects). It is the reason for the volatile prices of carmine and eliminates it from foods that are suitable for kosher, halal, vegetarian/vegan diets. This further means that carmine cannot be used in the food segments of plant-based foods⁹.

Thus, there exists a need in the industry for improved safe colorants which are heat and pH stable and which provide a bright intense color. This need is particularly strong within plant-based meat substitutes.

Talaromyces atroroseus biosynthesizes a class of novel pigments, called atrorosins. Atrorosins are “Monascus” like pigments, and have similar azaphilone scaffolds as the orange Monascus pigment PP-O, with a carboxylic acid group C-1 but are unique by their incorporation of amino acids into the isochromene system. Atrorosins are red pigments and their production is mycotoxin free. That distinguishes them clearly from Monascus pigments which are known to contain citrinin, which causes diverse toxic effects, including nephrotoxic, hepatotoxic, and cytotoxic effects and which excludes their use for industrial purposes in western countries. According to patent WO 2018/206590 A1, Atrorosins can be extracted with ethyl acetate (EtOAc) adjusted with formic acid and ammonium hydroxide, and further separated with semi-preparative HPLC using a C18 column. While this method is relatively simple, it is likely to have scalability issues regarding both throughput and costs, as preparative HPLC is a quite expensive unit operation compared to the industrial prices of food colorants. For food additives, purity and consistency of composition are important requirements. It is necessary to determine a specific degree of purity. This can be done by having a purity profile of the composition. In this profile, the ratio of active ingredient to impurities is determined. The impurities in a fermentation and extraction method as described in patent WO 2018/206590 A1 will typically include some by-products of the host organism incl. proteins, peptides, and organic acids, it can be carbohydrate residues from the growth medium, and it can be isomers of the active ingredient. For food manufacturers it is desirable to have a high purity degree with none or very low ratio of impurities, in order to ensure safety.

From an industrial perspective, atrorosins could serve as an alternative to betanin and carminic acid/carmine lake for food applications where these cannot fulfill the industrial requirements.

However, there is a need for a method to provide highly pure Atrorosin compositions in large quantities at low cost. The present invention provides such methods of producing, pure compositions and methods of using those.

### SUMMARY OF THE INVENTION

The aim of this invention is to provide improved compositions comprising Atrorosins as colorants for coloring foods, which are safe, have high stability towards pH and heat, and is capable of coloring foods with an intense red shade.

The invention solves the problem by providing scalable methods for making an atrorosin food coloring composition containing either atrorosin lake or atrorosin dye of high purity, with only small amounts of trans-atrorosin, and without the need for use of organic extraction solvents and preparative HPLC. The composition does not comprise any trace amounts of organic extraction solvents or other chemicals from HPLC preparation.

The invention provides a method for preparing an Atrorosin food coloring composition from fermentation broth, comprising the following steps:
a. Removing the biomass and other macro-sized constituents by membrane filtration and
b. Removing proteins, peptides and other constituents by ultrafiltration through an ultrafiltration membrane with a cut off between 1 kDa to 20 kDa, such as between 1 kDa and 100 kDa and
c. Acid precipitation of the Atrorosin of the permeate of step b and
d. Filtering the precipitate of step c by membrane filtration (with a membrane having a pore size below 1 μm)

For large scale synthesis purposes, and for the purpose of providing highly pure compositions to allow for use of the compositions in foods, new purification methods for atrorosins needed to be developed. It was surprisingly found by the inventors that such pure atrorosin compositions could be made by initial membrane filtration and ultrafiltration steps to remove macro-sized constituents, proteins and peptides, and subsequent acid precipitation to provide highly pure precipitates, mainly consisting of the atrorosin.

The invention further provides a method to make water soluble Atrorosin powder comprising an Atrorosin salt and salt of a buffer. Such powder is useful for making dyes and lakes. The method for making the water soluble Atrorosin powder based on the precipitate of the above step d. includes:
e. Adding a buffer (e.g. citrate buffer) to increase the pH to above the pKa of the Atrorosin of the precipitate to increase its water solubility and
f. Drying step to remove water to make an Atrorosin powder comprising a salt composition of Atrorosin and salt of the buffer

The atrorosin powder, precipitate or composition provided by the method of the invention differ in more than one important parameter from the atrorosin provided by the previously known methods. The atrorosin powder, precipitate or composition provided by the invention does not comprise significant amounts of trans-atrorosin and does not comprise proteins, peptides, and organic acids, or carbohydrate residues from the growth medium. Thus, the invention provides novel compositions made by the method of the invention.

The invention further provides uses of the compositions of the invention, comprising Atrorosin, as a colouring agent and/or for preservative purposes for any one of a food, a non-food product and a cosmetic. In some embodiments, the invention provides products comprising the Atrorosin pigment such as food, a non-food products and cosmetics. In some embodiments kits for coloring and/or for preserving a product, wherein the kit comprises at least one Atrorosin pigment according to the invention, and wherein the pigment is supplied in a container, and wherein the product is selected from a food, a non-food and a cosmetic. In some embodiments, the food is a dairy product, or a food that is mixed with oil or fat, or one that has a low pH, or where the food will be heated, or is made for heating before eating. In some embodiments, the food is a meat substitute or a beverage.

In some embodiments, the invention provides a lake or a dye made by the compositions made by the method of the invention. In all these embodiments and uses, Atrorosin lake or dye of the invention has advantageous stability properties, whether the composition will be used at high temperatures, low pH, light exposure for a long time, or will be stored for a long time.

### FIGURES

FIG. 1. Powder produced in step f. of the method of the invention, and as described in example 2.

FIG. 2. Improved purity profile of the atrorosin of the invention (marked “New Invention”), when compared with the Atrorosin prepared by the HPLC method of WO 2018/206590 A1. The BPC is the base peak chromatogram which detects all components of the sample, whereas the UV/VIS (520 nm) only detects components with an emission at 520 nm. As Atrorosin-E in the experiment is here in a solution of methanol and acetonitrile the peak has shifted from 490 nm to 520 nm. The preparative HPLC clearly binds other constituents from the ethyl acetate extract, whereas the invention with filtration steps and acid precipitation removes most impurities that bind to the HPLC. The level of trans-isomer is much higher in the composition made by the preparative HPLC method as compared with the acidic conditions.

FIG. 3. Antioxidant activity of atrorosinE measured by DPPH assay.

FIG. 4. pH stability test of the atrorosin powder of step f. step 0 is without atrorosin, step 1-3 is with atrorosin. Step 1 is at pH 1.5, step 2 is at pH 10, step 3 is at pH 5.

FIG. 5 i. Burger patty, where A) Is just the base of vegan burger patty, B) Has added 0.05% atrorosin dye and C) Has added 0.1% atrorosin dye.

FIG. 5 ii. Burger patty, as in 5 I, wherein the burger patty is fried.

### DETAILED DESCRIPTION OF THE INVENTION

The present invention provides a method for producing highly pure Atrorosin compositions. The method of the invention is a scalable method for making compositions comprising Atrorosin at low cost. The invention further provides compositions comprising atrorosin made by the method, as well as uses of the compositions, products and kits comprising the compositions. In preferred embodiments, the products are foods, non-food and cosmetics.

Present alternative marketed coloring compositions have weaknesses relating to market demands, as they are either not vegan, not heat stable above 40° or not stable at low pH, or inefficient production methods means that high volumes cannot be provided on a continuous basis and at low cost. The atrorosin compositions provided by the method of the invention fulfill all these criteria.

In some embodiments, the uses of the compositions provided by the present invention will be under acidic or heated conditions, or both. In some embodiments, the compositions are for use as colorant in food products, such as i.e. dairy products or meat substitutes. Many dairy products are acidic, and it is important for the consumer that colorant used in the food to improve appearance is stable even at low pH. Furthermore, prior to use, many foods are heated. Such foods include meat substitutes. Therefore, it is also important that any colorants used to improve colouring of such foods are stable even when heated. In some embodiments, the compositions provided by the method of the invention are for use as a colouring agent in foods, non-foods and cosmetics. In some embodiments, the compositions of the invention are formulated as a lake or a dye. The compositions of the invention are highly pure, i.e. they only comprise low levels of trans-atrorosin in contrast to atrorosin purified by HPLC. Furthermore, the compositions of the invention does not comprise significant amounts of proteins, peptides, and organic acids, or carbohydrate residues from the growth medium.

The inventors of the present invention has found that the atrorosin compositions of the invention are antioxidants, and thus the invention provides use of the compositions of the invention as antioxidants. In some embodiments, the use as antioxidant is as a preservative for food or cosmetics. In some embodiments, the use is use of atrorosin according to the invention as a medicament of for cosmetic use, as an anti-ageing composition, for preventing cancer, heart disease, or for ameliorating radiation damage, or for preventing diseases or conditions where antioxidants are helpful.

### Terms

Colorant: A colored substance (molecules) that is either a dye or a pigment.

Dye: Dyes are colored substances (molecules) that are soluble in the liquid/medium which they are mixed into. In general, dyes are soluble in water.

Pigment: Pigments are colored substances (molecules) that do not dissolve in liquid/medium with which they are mixed. In general, pigments are insoluble in water.

Atrorosin: is a colorant having the chemical formula CQ3HQ406NR, where NR is a compound containing a primary amine, such as an amino acid, and the configuration of the double bond between carbon 2 and 3 is cis.

In some embodiments, the atrorosin has the structure of Formula I:

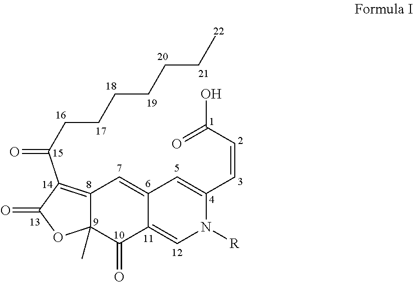

wherein N—R is selected from the group consisting of an amino acid, a peptide, an amino sugar and a primary amine, and the configuration of the double 5 bond between carbon 2 and 3 is c/s.

In some embodiments, the atrorosin pigment is of Formula I, wherein N—R is selected from among an amino acid, a peptide, an amino sugar and a primary amine, and the configuration of the double bond between 5 carbon 2 and 3 is c/s, wherein said amino acid is selected from one of the group consisting of: L-alanine, L-arginine, L-asparagine, L-aspartate, Lcysteine, L-glutamate, L-glycine, L-histidine, L-isoleucine, L-leucine, L-lysine, L-methionine, L-phenylalanine, L-serine, L-threonine, L-tyrosine, L-valine and L-ornithine.

According to one embodiment, the invention provides an atrorosin pigment, or a composition or kit comprising atrorosin having the structure of Formula I as defined above, and wherein the methods of the invention are part of the process of making the compositions.

WO 2018/206590 A1 present a method for making and purifying atrorosin pigment. However, the compositions provided by the method of WO 2018/206590 A1 are expensive to produce and not sufficiently scalable. Furthermore, the method of WO 2018/206590 A1 produces compositions comprising unwanted contaminant compounds, including large amounts of trans-atrorosin. Thus, improved scalable methods are needed, which are capable of making compositions comprising atrorosin having improved purity as compared to the previously known method. The method of the present invention is less expensive in use for purification than the method of WO 2018/206590 A1, and provides a scalable method for preparing atrorosin food coloring compositions of improved purity. The method has been tested for purification of atrorosin from 5 liter culturing batches as well as from 50 liter batches with good results regarding all the parameters. The results as described in examples 1 and 2 respectively shows that the method works well for purification of atrorosin from batches of increasing sizes. The method of the invention provides means for removing unwanted macro-sized constituents, proteins and peptides originating from the fermentation steps, the removal of which are needed in order to make the compositions suitable for use as food ingredients. Further, unlike the HPLC purification method of WO 2018/206590 A1, the method of the invention cause almost no formation of trans-atrorosin. In some embodiments, the methods of the invention provides compositions with less than 20% by weight trans-atrorosin, such as less than 10% by weight trans-atrorosin, such as less than 5% by weight trans-atrorosin. In some embodiments, the atrorosin is atrorosinE, such as atrorosinE wherein less than 20%, such as less than 10%, such as less than 5% by weight is trans-atrorosinE. The method of the invention further provides compositions wherein less than 10% by weight is proteins or peptides, such as less than 5%, such as less than 4% or less than 3% or less than 2% or less than 1% is protein or peptides.

The method comprises steps for preparing highly pure Atrorosin food coloring compositions from fermentation broth, including the following steps:
a. Removing the biomass and other macro-sized constituents by membrane filtration and
b. Removing proteins, peptides and other constituents by ultrafiltration through an ultrafiltration membrane with a cutoff between 1 kDa to 20 kDa and
c. Acid precipitation of the Atrorosin of the permeate of step b and
d. Filtering the precipitate of step c by membrane filtration (with a membrane having a pore size below 1 μm) and
e. Adding a buffer to increase the pH to above the pKa of the Atrorosin of the precipitate to increase its water solubility (e.g. citrate buffer) and
f. Drying step to remove water to make an Atrorosin powder comprising a salt composition of Atrorosin and salt of the buffer

Starting from fermentation broth derived from the cultivation of Talaromyces atroroseus which may be produced according to the methods described in WO 2018/206590 A1, the biomass and other macro-sized constituents of the broth are removed by a filtration step using conventional membrane filtration with a pore size between 1-40 μm. The resulting permeate is then filtered through an ultrafiltration membrane with a cutoff between 1 kDa to 20 kDa, such as between 1 kDa and 100 kDa, to remove proteins, peptides, and other constituents. The Atrorosins in the resulting permeate can then be acid precipitated with strong acids, by lowering the pH of the permeate to be lower than the pKa of the Atrorosin. In some embodiments, the atrorosin is Atrorosin-E, and the pH is lowered to be below pH 1,7, such as between 1.7 and 0.5. By strong acids are meant acids which is virtually 100% ionized in solution. In some embodiments, acids useful for the precipitation step have a pKa below about 3, such as below about 2.5, such as below about 2,3. Acids useful for the precipitation step includes but are not limited to hydrochloric acid, nitric acid, phosphoric acid, and sulfuric acid. The following precipitate is then be collected by another membrane filtration step. The precipitates have a size of 1-5 μm so membrane filters below this particle size are useful for the collection step. The collected precipitate has a low pH and are not dissolvable in water, but it can be dissolved by increasing the pH to higher than the pka. For foods, a citrate buffer is well approved, and a low buffer solution at pH 6 will dissolve all the atrorosin precipitate. A large number of buffers are suitable for use in foods and cosmetics. Such buffers include but are not limited to Phosphate buffer: (50% 1M K2HPO4 & 50% 1M KH2POH4), Phosphate buffered Saline, PBS (8 g/L NaCl, 0.2 g/L KCl, 1.44 g/L Na2HPO4, 0.24 g/L KH2PO4) and Tartrate buffer. Further suitable buffers may readily be identified by the skilled person without undue burden, and are included by the scope of the present invention. The resulting liquid comprising the dissolved atrorosin and buffer, can then be made to powder by removing water by e.g. lyophilizing, spray drying, evaporation etc. The resulting powder is an atrorosin salt composing of atrorosin and e.g. sodium citrate. Another advantage of the present invention is that the purity profile of the salt is much better than after ethyl acetate extraction and preparative HPLC. As compared with the known HPLC preparation method, the method of the present invention provides atrorosin compositions having much less unwanted impurities, and the level of trans-isomers is much lower in the present invention. Data comparing trans-atrorosin content of compositions comprising HPLC purified atrorosin with compositions comprising atrorosin purified with the present invention are presented in example 3.

Using the method of the invention, the obtained atrorosin powder is made without the use of organic extraction solvents and preparative HPLC, and only by inexpensive unit operations already implemented in food ingredient production facilities. This method is faster, less labor consuming, and cheaper than the extraction method of patent WO 2018/206590 A1.

In some embodiments, the filtration of step a. is done using a filter with a pore size of between 1 μm and 40 μm. In some embodiments, the pore size of the filter is between 1 and 20 μm, and in some embodiments the pore size is between 20 and 40 μm.

In some preferred embodiments, the acid precipitation in step c. is done by lowering the pH of the permeate to be lower than the pKa of the Atrorosin. Thus, the invention provides a means for removing macroconstituents from the fermentation process, and in a subsequent precipitation step for isolating the atrorosin. The inventors have found that using the method of the invention, purification of atrorosin can be done without the formation of trans-atrorosin and without the use of organic solvents, which would have been present in the final composition if purification was by the previously known HPLC purification method. Example 3 present data demonstrating the increased purity with regard to trans-atrorosin content of the composition made by the method of the invention as compared to a HPLC purified atrorosin composition.

In some preferred embodiments of step c., atrorosin is precipitated by lowering the pH of the permeate from step b. to be below the pKa of atrorosin, this is done by addition to the permeate of strong acids such as hydrochloric acid, nitric acid, phosphoric acid or sulphuric acid.

In some embodiments of step c., atrorosin is precipitated by lowering the pH of the permeate from step b. to be below the pKa of the atrorosin by addition of an acid selected from the list of Hydroiodic (HI), Hydrobromic (HBr), Perchloric (HClO4), Hydrochloric (HCl), Chloric (HClO3), Sulphuric (1) (H2SO4), Nitric (HNO3), Hydronium ion (H3O+), Iodic (HIO3), Oxalic (1) (H2C2O4), Sulphurous (1) (H2SO3), Sulphuric (2) (HSO4-), Chlorous (HClO2), and Phosphoric (1) (H3PO4).

In some embodiments, the methods of the invention comprises a filtration step to isolate the acid precipitate, followed by a step to raise the pH with a buffer to increase water solubility of the precipitate. In one embodiment, the buffer of step e. is a citrate buffer. In some embodiments, the buffer is a buffer suitable for use in food or cosmetic products. In some embodiments, the solubilised atrorosin of step e. is dried to make an atrorosin powder. In some embodiments the drying is by lyophilisation, spray drying, or by evaporation.

In some embodiments, the method of the invention comprises the following steps:
a. Removing the biomass and other macro-sized constituents by membrane filtration using a filter with filter size 1-40 μm and
b. Removing proteins, peptides and other constituents by ultrafiltration through an ultrafiltration membrane with a cutoff between 1 kDa to 20 kDa, such as between 1 kDa and 100 kDa and
c. Acid precipitation of the Atrorosin of the permeate of step b by lowering the pH to be below the pka of the Atrorosin, by using a strong acid, such as an acid selected from any of hydrochloric acid, nitric acid, phosphoric acid or sulphuric acid and
d. Filtering the precipitate of step c by membrane filtration (with a membrane having a pore size below 1 μm) and
e. Adding a buffer to increase the pH to above the pKa of the Atrorosin of the precipitate, such as to a pH below 10, such as a pH between 5 and 10, such as a pH between 5 and 8, such as about pH 7, to increase its water solubility. The buffer may in this embodiment be selected from any one of a citrate buffer, a Phosphate buffer: (50% 1M K2HPO4 & 50% 1M KH2POH4), Phosphate buffered Saline, PBS (8 g/L NaCl, 0.2 g/L KCl, 1.44 g/L Na2HPO4, 0.24 g/L KH2PO4) and Tartrate buffer, or another suitable buffer easily selected by the skilled person and
f. Drying step by use of lyophilisation, spray drying, or by evaporation to remove water to make an Atrorosin powder comprising a salt composition of Atrorosin and salt of the buffer.

In some embodiments, the invention provides an atrorosin salt obtainable by the method according to steps a-d. In some embodiments, the invention provides the powder obtainable by the method according to the methods of the invention, i.e. by steps a-f. In some preferred embodiments, the atrorosin is atrorosin-E, wherein N—R is L-glutamate. In some preferred embodiments the salt or the powder of the invention comprises Atrorosin which is Atrorosin-E. In some embodiments, the atrorosin of the invention is an atrorosin where the N—R is an amino acid selected from one of the groups consisting of: L-alanine, L-arginine, L-asparagine, L-aspartate, L-cysteine, L-glutamate, L-glycine, L-histidine, L-isoleucine, L-leucine, L-lysine, L-methionine, L-phenylalanine, L-serine, L-threonine, L-tyrosine, L-valine and L-ornithine.

Food additives and ingredients for cosmetics need to have a consistent and reproducible high purity, and therefore, a method for its production must be reliable with regard to reproducibility of the quality of the product made. Furthermore, methods for producing such compounds must be scalable to allow production of any amount needed at attractive pricing. Evaluation of the suitability of a compound or composition for use as food additive or in cosmetics, include assessment of the risk of potential harmful effects to the end user. Characterization of genotoxicity, oral bioavailability and temperature and pH stability are included in the assessment. Food and cosmetics additives such as colorants must be free of genotoxicity, have low oral bioavailability and be stable at varying temperature and pH. In order to be able to trust such data, the compound or composition need to be consistent from batch to batch. The methods of the invention allows for consistent production of very high purity atrorosin. In some embodiments, the compostions according to the invention are composed of at least 80% by weight of cis-Atrorosin, and less than 20% trans-Atrorosin. In preferred embodiments, the atrorosin is atrorosin-E, wherein at least 80% is cis-atrorosin-E. In some embodiments, at least 80% by weight, such as at least 85%, 90%, 95% or 96%, 97%, 98% or at least 99% by weight is cis-atrorosin, such as cis-atrorosin-E. In some embodiments, less than 15%, such as less than 10%, such as less than 5%, such as less than 4%, 3%, 2% or less than 1% by weight is trans-atrorosin such as trans-atrorosin-E. In some embodiments, impurities such as proteins or peptides are removed by the methods of the invention, such that the compositions comprises less than 10% by weigh of proteins or peptides, such as less than 5%, such as less than 4% or less than 3% or less than 2% or less than 1%.

The atrorosin salt can readily be formulated into either a dye with carbohydrates, proteins or oligosaccharides such as in non-limiting example, with maltodextrin, sucrose, saccharose, cellulose, cyclodextrin, pectin, starch, chitin, lactose, or maltose, or with proteins such as in non-limiting example soy protein or whey protein. Example 7 demonstrate formulation of atrorosin salt into a dye by mixing atrorosin with maltodextrin. Example 7 describe formulation of an atrorosin dye with maltodextrin. Similar to this, the other carbohydrates, proteins or oligosaccharides may be used to make atrorosin dyes. Pigment lakes, can be made from the atrorosin salt, by coupling it to aluminum or other metals such as calcium, barium, zinc, sodium or copper.

In some embodiments, the invention provides a use of the salt, the powder or the composition according to the invention for producing a lake. In some embodiments, Atrorosin salt, powder or composition is used for producing a lake by coupling to a metal. In some embodiments, producing the lake is by coupling the Atrorosin salt, powder or composition to anyone of aluminium, calcium, barium, zinc, sodium or copper. Example 8 demonstrate lake formation with aluminium. Lake formation with the other metals may be in a similar manner as described in example 8. In some embodiments, the lake comprises atrorosin-E.

In some embodiments, the lake is made by mixing atrorosin and mordant (the metal) in a weight to weight relationship within the range of 1:1 to 1:20 where 1 is atrorosin and 20 is the mordant. In preferred embodiments, the atrorosin:mordant relationship is in the range between 1:3 to 1:6 w/w. The lake produced in example 8 has a 1:6 atrorosin:mordant relationship.

In some embodiments, the invention provides a dye comprising the salt, the powder, or the composition comprising Atrorosin. In some embodiments, the dye is an Atrorosin formulation with sugars, or other carbohydrates. In some embodiments, the carbohydrate of the previous embodiment is anyone of maltodextrin, sucrose, saccharose, cellulose, cyclodextrin, pectin, starch, chitin, lactose, or maltose, or the dye is formulated with proteins such as in non-limiting example soy protein or whey protein. In some embodiments, the carbohydrate of the dye is anyone of maltodextrin, cyclodextrin or pectin. In some embodiments, the dye comprises atrorosin-E.

The atrorosin of the present invention is highly stable at low pH and at high temperatures. Example 9 demonstrate atrorosin stability at various pH. Example 10 demonstrate stability at high temperature. It is clear from the data in these examples that atrorosin shows superior pH stability when compared with carminic acid, and superior temperature stability in the 60-80 degree C. range as compared with betain.

The invention further provides uses of the highly pure, heat and pH stable Atrorosin compositions, precipitates or powders for making dye and lake compositions for coloring of foods, non-food products and cosmetics. pH and temperature stability is important for the contemplated industrial use of the atrorosin dyes and lakes. The data provided in the examples regarding pH and temperature stability, were in aqueous buffer solutions, and thus relevant for the contemplated uses in foods.

Antioxidant properties are beneficent for coloring compositions. Antioxidants prevent or inhibit oxidation processes and are thought to have both health benefits in relation to heart conditions and cancer, but also preservative effects elongating the shelf life of foods, especially when mixed with fat or oils. DPPH Inhibition was used to demonstrate antioxidant activity of the atrorosin compositions of the invention. Example 11 present data demonstrating the antioxidant effects of atrorosins.

Thus in some embodiments, the invention provides the use of an Atrorosin pigment according the invention, such as the salt, powder, dye or lake, as a colouring agent and/or as an antioxidant, and/or for preservative purposes for any one of a food, a non-food product and a cosmetic. In some embodiments the cosmetics is a lipstick. Lipstick users ingest some of the lipstick applied to the lips, why it is advantageous that the dyes or lakes used for colouring the lipstick only has a very low bioavailability when given orally. Antioxidants are often components of lipsticks and other cosmetics for anti-ageing purposes, and for preservation. Therefore, the findings of the inventors (see example 11) that atrorosins has a very low bioavailability when administered orally is a great advantage which makes it particularly suitable for use in cosmetics such as in lipstick. The finding that atrorosin is not genotoxic (example 12) is also an important finding that further supports its use in food and cosmetics such as in lipstick. In some embodiments, the atrorosins or the atrorosin compositions of the invention is for use in anti-ageing compositions, such as in anti-ageing cosmetic products. In some embodiments the atrorosin compositions of the invention are for use in health products for prevention of heart disease or cancer, or for amelioration or treatment of radiation damage.

In a further embodiment, the invention provides a product comprising the Atrorosin pigment according to the invention, wherein the product is selected from a food, a non-food product and a cosmetic, such as in non-limiting example, a lipstick or an anti-ageing composition for cosmetic use.

In some embodiments, the invention provides a kit for colouring and/or for preserving a product, and/or for making a cosmetic, such as a lipstick or an anti-ageing composition. In some embodiments, the kit comprises at least one Atrorosin pigment according to the invention, wherein the pigment is supplied in a container further comprising instructions for use, and wherein the product is selected from a food, a non-food and a cosmetic.

Dairy products often comprise ingredients such as jam or fruit, and in order to provide a product with an attractive colour, it may sometimes be advantageous to add some additional colour. In some embodiments, the invention provides for the use of the atrorosin pigment of the invention, product comprising the atrorosin pigment, or kit according to the invention, wherein the product is a food and the food is a dairy product. The pH stability of the atrorosin of the invention is well suited for use in dairy products, even when the dairy product has a low pH.

In some embodiments, the use, product or kit according to the invention is wherein the product is a food, such as a dairy product. In some embodiments, the use, product, or kit according to the invention is wherein the product is a food mixed with oil or fat.

Many dairy products or other food products are acidic, and due to excellent pH stability properties of the Atrorosins of the present invention, use of the compositions of the invention in such products are among the preferred uses. Thus in some embodiments, the use, product and kit according to the invention, is for food products wherein the product is a food having a pH below 7, such as a pH below 6, such as below pH 5, such as below 4, such as below 3.

The atrorosin of the invention also has excellent heat stability and the colouring component of the compositions is non-toxic. The presented invention describes that atrorosin is neither genotoxic nor does it have any adverse effects on rats (see results of example 12. Atrorosin-E has a very low bioavailability (0.3%), has a moderate plasma clearance (16±2 mL/min/kg) and has a half-life of only 1.3±0.6 hours, and thus is well suited for use in foods or other products that is heated. In some embodiments, the invention provides for use, products and kit according to the invention, wherein the product is a food for heating subsequent to the addition of the Atrorosin pigment.

In some embodiments, the atrorosin according to the invention is for use in foods heated to at least 50 degrees C., such as at least 60, 70, 80, 90, 100, 150 or at least 200 degrees C. In some embodiments, the use is for colouring of meat substitutes, such as in non-limiting example, a burger patty. In some embodiments, the use, is for colouring a product which is a beverage. In some embodiments, the invention provides a product coloured by the atrorosin of the invention, wherein the product is a food, a non-food or a cosmetic.

In some embodiments, the invention provides a food such as a meat substitute or a beverage comprising the atrorosin composition, powder or salt, lake or dye of the invention.

In some embodiments, the invention provides a use of the atrorosin according to the invention, a kit or a product according to the invention, wherein the product is acidic and is for heating.

In some embodiments, the atrorosin compositions are for use together with other pigments, antioxidants or preservatives. In some embodiments, the invention provides a product, such as a food, a non-food, or a cosmetic, wherein the product comprises atrorosin in combination with other pigments, antioxidants or preservatives.

### Description of Laking:

A pigment lake, is a pigment where an organic dye is fixed to a metallic salt, rendering it insoluble. Metallic salts are typically colorless, so the color of the organic component will determine the color of the precipitate lake. For foods, the most commonly used metallic salt is alumina hydrate (aluminum hydroxide). The color lake pigments will have different technical properties compared to the organic dye, many of these resulting from the metallic salt which it is bound to. Besides being made insoluble, the lake can also have improved pH stability, temperature stability or oxidation stability. FD&C lakes (the US FDA approved lakes) are oil dispersible but not oil soluble making them useful for mixing with oils and fats, which the dye counterpart would not be able to. Lakes can be in specific concentrations depending on the dye and amount of dye in the laking process.

In some embodiments, the lake is made by mixing atrorosin and mordant (the metal salt) in a weight to weight relationship within the range of 1:1 to 1:20 where 1 is atrorosin and 20 is the mordant. In preferred embodiments, the atrorosin:mordant relationship is in the range between 1:3 to 1:6 w/w. The lake produced in example 8 has a 1:6 atrorosin:mordant relationship.

### Description of Encapsulation:

Microencapsulation is a defined technology where solids and liquids are packaged into sealed capsules of sizes between nanometers and millimeters. The encapsulation matrix can improve certain technical abilities, such as stability towards oxidation, temperature or to improve the dispersibility of pigments in water. The packaged material is called the active material while the packaging material is called the shell. Microencapsulation creates a physical barrier between the active material and the outside environment of the shell, thereby potentially increasing the stability against ambient conditions. There are many techniques to microencapsulate but the main techniques are spray-drying, freeze-drying, coacervation and emulsion. The most used shell materials gums like gum Arabic, low molecular weight carbohydrates like maltodextrin, saccharose, dextrin, cellulose, gelatin, lipids and proteins like soy proteins.

### Description of Meat Substitutes, and Laboratory Meats:

A meat substitute are foods which approximates the aesthetics of specific types of meat (such as texture, flavor, appearance). They typically have a protein base made from vegetarian or vegan ingredients such as soy-based (tofu, tempeh), gluten based, or pea based. It is therefore increasingly important to only use vegetarian and vegan additives. The increasing focus and demand for sustainable diets has made meat substitutes an emerging market. The challenge for many meat substitute producers is to make the alternative meat look and taste as close as possible to real meat both pre and post cooking. Betanin and anthocyanins are currently used in a complex mixture however the colors are more pinkish before cooking and mostly lost after cooking.

Laboratory meat or cultured meat, is in vitro cell culture of animal cells. Growth and proliferation of this happens inside a laboratory inside a bioreactor before meat is harvested. Heme proteins are both important for proliferation of cells however they are also used to induce a color change to more closely resemble traditional meats.

### Expected Purity Requirements:

Based on the process described in this invention, the composition will atrorosin will be 90% cis atrorosin-E, with less than 3% trans atrorosin-E. The composition will have less than 5% carbohydrates, such as sucrose, glucose, and fructose remaining from the fermentation media. The composition will have unidentified impurities will have a less than 5% by weight. The composition is substantially free of Talaromyces atroroseus proteins, which means that they are not detectable by SDS-PAGE analysis.

### List of Foods:

In non-limiting example, the atrorosin compositions of the present invention may be used for coloring of Meat (sausages etc.), meat substitutes (pea based etc. as described above) and confectionary (typically foods by sugar or with high sugar content) (for example Cakes, pastries, cookies, but also candy, gum, chocolates and candied nuts), and dairy products such as in non-limiting example yogurts, ice creams, etc.

### EXAMPLES

### Example 1: Production of Atrorosin Salt in Small Scale

5 L of fermentation broth (produced according to the methods of WO2018206590), containing approximately AU₄₉₀ of 12 of Atrorosin-E pigment, was filtered through miracloth to remove biomass and other macro-sized constituents, resulting in 4 L of permeate containing approximately AU₄₉₀ of 10 of Atrorosin-E pigment. The 4 L of permeate was further filtered through an ultrafiltration membrane with a cut-off of 10 kDa, to remove majority of proteins and peptides. The 3.5 L permeate contained approximately AU₄₉₀ of 7 of Atrorosin-E pigment.

The pH of the permeate was lowered to a pH of 1.34 with addition of 60 mL of 5 M HCl. The permeate was stored at 5° C. for 24 hours with stirring. During this time, the Atrorosin-E pigment precipitated. After 24 hours, the mixture was filtered through a 0.8 μm cellulose membrane. The retentate was collected and dissolved in 2 L of 20 mM citrate buffer pH 6. This resulting mixture contained approximately AU₄₉₀ of 13 of Atrorosin-E pigment. The mixture was frozen prior to lyophilization. 9.5 g of resulting powder was collected and measured to have an E1% of 20.

In the context of the examples E1% is:

E1% is a comparison of tinctorial strength

Absorbance of 1% (10 g/L) of product at a specific absorbance, where this is typically the maximum absorbance wavelength in nm. Maximum absorbance wavelength can be dependent on which medium the colorant is dissolved in.

For pure colorants, the E1% absorbance relates to the molar absorptivity in the following:

E^{1%}_{cm}=10*ε/M

### Example 2: Production of Atrorosin Salt in Pilot Scale

Approximately 50 L of fermentation broth containing approximately AU₄₉₀ of 25 of Atrorosin-E pigment, was filtered through miracloth to remove biomass and other macro-sized constituents, resulting in 35 L of permeate containing approximately AU₄₉₀ of 23 of Atrorosin-E pigment. The 35 L of permeate was filtered through an ultrafiltration membrane with a cutoff of 10 kDa, to remove the majority of proteins and peptides. The 30 L permeate contained approximately AU₄₉₀ of 17 of Atrorosin-E pigment.

The pH of the permeate was lowered to a pH of 0.95 with addition of 700 mL of 5 M HCl. The permeate was then stored at 5° C. for 24 hours. After 24 hours, the mixture was filtered through a 0.8 μm cellulose membrane. The retentate from the filtering was then collected and dissolved in 4 L of 10 mM citrate buffer pH 6. This resulting mixture contained approximately AU₄₉₀ of 72 of Atrorosin-E pigment. The mixture was frozen prior to lyophilization. 25 g of resulting powder was collected measured to have a E1% of 88.

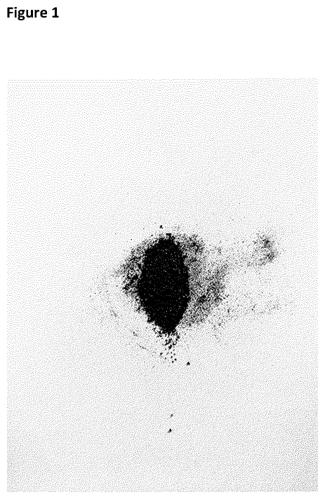

FIG. 1 shows powder produced in step f of the method of the invention, and as described in example 1.

### Example 3: Purity of Atrorosin Salt

Purity is an important necessity of the coloring composition.

Purity of the resulting powder from example 2, was assessed using Ultra-high Performance Liquid Chromatography-High Resolution Mass Spectrometry (UHPLC-HRMS) was performed on an Agilent Infinity 1290 UHPLC system (Agilent Technologies, Santa Clara, Calif., USA) equipped with a diode array detector. Separation was obtained on an Agilent Poroshell 120 phenyl-hexyl column (2.1×250 mm, 2.7 μm) with a linear gradient consisting of water and acetonitrile both buffered with 20 mM FA, starting at 10% B and increased to 100% in 15 min where it was held for 2 min, returned to 10% in 0.1 min and remaining for 3 min (0.35 ml/min, 60° C.). An injection volume of 1 μL was used.

MS detection was performed in positive detection mode on an Agilent 6545 QTOF MS equipped with Agilent Dual Jet Stream electrospray ion source with a drying gas temperature of 250° C., gas flow of 8 L/min, sheath gas temperature of 300° C. and flow of 12 L/min. Capillary voltage was set to 4000 V and nozzle voltage to 500 V. Mass spectra were recorded at 10, 20 and 40 eV as centroid data for m/z 85-1700 in MS mode and m/z 30-1700 in MS/MS mode, with an acquisition rate of 10 spectra/s. Lock mass solution in 70:30 methanol:water was infused in the second sprayer using an extra LC pump at a flow of 15 μL/min using a 1:100 splitter. The solution contained 1 μM tributylamine (Sigma-Aldrich) and 10 μM Hexakis (2,2,3,3-tetrafluoropropoxy) phosphazene (Apollo Scientific Ltd., Cheshire, UK) as lock masses. The [M+H]+ ions (m/z 186.2216 and 922.0098 respectively) of both compounds was used.

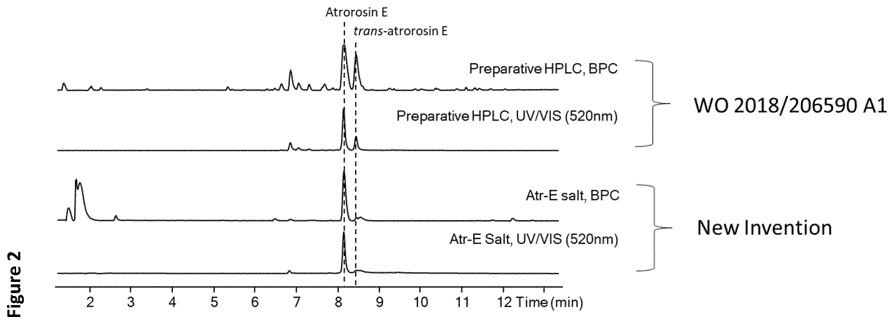

FIG. 2 shows the improved purity profile of the atrorosin of this new invention as compared with the atrorosin prepared by the method of WO 2018/206590 A1. In particular, the great reduction in amount of trans-atrorosin is noted. The BPC is the base peak chromatogram which detects all components of the sample, whereas the UV/VIS (520 nm) only detects components with an emission at 520 nm. As atrorosin-E is here in a solution of methanol and acetonitrile the peak has shifted from 490 nm to 520 nm. The preparative HPLC clearly binds other constituents from the ethyl acetate extract, whereas the invention with filtration steps and acid precipitation removes most impurities that bind to the HPLC. The level of trans-isomer is also much higher in the preparative HPLC method as the acidic conditions while dissolved in methanol favors for isomerization, while this does not happen in aqueous solutions.

### Example 4

### Quantification of Non-Coloring Composition.

10 mg of powder from example 2, can be injected into a semi-preparative HPLC, and fractionated based on the UV 520 nm signal. This will give to major fractions of either red colored components where Atrorosin-E is the major constituent, and a second fraction with the remaining constituents. These two fractions can then be dried using a rotary evaporator and the percentage of coloring constituents can then be calculated by weighing the total red color compared to the total amount of dry matter.

### Example 5

### Quantification of Proteins

To determine that Atrorosin-E is substantially free of proteins from Talaromyces atroroseus, an SDS-PAGE analysis may be used. Conventional SDS-PAGE analysis can detect proteins and peptides down to a size of 3,000 Da.

Running solvent is a Tris Buffer pH 8, (20 g tris/1000 ml demineralized water adjusted with HCl.

Sample of Atrorosin-E to be tested in a concentration of 1.5 g/L. 1M of DTT was added to samples and markers. 10 μL of sample and markers are added to the pre-cast NUPAGE Novex high performance gel 4-12% BIS-TRIS and stained by either comassie blue or silver staining.

### Example 6

### Quantification of Carbohydrates

Remaining carbohydrates such as sucrose, glucose, fructose etc. can be detected and quantified by HPLC. A sample of 1.0 g/L of powder from example 2, can be run on HPLC system with an Aminex HPX-87H cation-exchange column (BioRad, Hercules, Ca, USA). Compounds are separated using an isocratic elution at 30° C. with 5 mM H₂SO₄. Quantification of standards is performed using a six-level calibration curve with glucose and pyruvate detected at wavelength 210 nm and sucrose, fructose, succinate, glycerol, acetate, and ethanol by refractive index.

Based on the calibration, carbohydrates and small acids can be quantified in the Atrorosin-E sample.

### Example 7: Formulation of Atrorosin with Maltodextrin

1 gram of the resulting powder from Example 2, was dissolved in 20 ml of water and stirred for 5 minutes. 3 grams of maltodextrin was slowly added to mixture the while stirring. After the maltodextrin was fully dissolved, the mixture was frozen prior to lyophilization. 3.8 grams was recovered, and it was measured to have an E1% of 21.

### Example 8: Formulation of Atrorosin with Aluminum as a Lake

1 gram of the resulting powder from Example 2 was dissolved in 200 ml of water and stirred for 5 minutes, with an AU₄₉₀ of 100. 100 ml of Aluminum potassium sulfate (AlK(SO₄)₂*12H₂O) 1M was prepared by dissolving 47.4 g of AlK(SO₄)₂*12H₂O in 100 ml of water and heated to 80° C. with stirring. 100 ml of Sodium carbonate (Na₂CO₃) 1M was prepared by dissolving 10.6 g of Na₂CO₃ in 100 ml of water and stirred.

An Aluminiumhydroxid (Al(OH)₃) solution was prepared by addition of 20 ml of 1M Na₂CO₃ to 20 ml of 1M AlK(SO₄)₂*12H₂O and 1.2 ml of 5M NaOH to give the final solution a pH of 10.

Aluminum lake was prepared by mixing Atrorosin 10:1 with the aluminiumhydroxid solution. To 200 ml of atrorosin containing liquid of 5 g/L, 20 ml of Aluminiumhydroxid was added and pH was adjusted to pH 4 with 5M HCl and 5M NaOH. The slurry was incubated at 50° C. for 1 hour, before it was set to cool at room temperature overnight. This gives an approximate atrorosin to aluminiumhydroxid relationship of 1:6 by weight in the lake.

After cooling overnight, the slurry was filtered on a Whatman filter Grade 50. Atrorosin aluminum lake was dried at 50° C. for 2 hours. Uncoupled slurry was collected and had an AU₄₉₀ of 9, meaning that 91% of atrorosin was coupled to aluminum. After drying, atrorosin lake was scrapped off the filter and grinded into fine powder by mortar and pestle.

### Example 9: pH Stability of Atrorosin Compared to Carminic Acid

100 mg of resulting powder from example 2, was dissolved in 250 ml in McIlvaine buffer ranging from pH 2.2-7.2. Buffer and atrorosin was mixed for 5 minutes before the solutions were subjected to colorimetric analysis using spectrophotometer. The absorption maxima at 425 nm and 490 nm were determined in a microtiter plate with a 0.45 cm cell length with buffer as blank.

Absorption spectrum of Atrorosin-E at different pH values is shown in table 1.

TABLE 1

| Wavelength (nm) | pH 2.2 | pH 3.5 | pH 5 | pH 7.2 |
|---|---|---|---|---|
| 425 | 0.63 | 0.73 | 0.75 | 0.73 |
| 490 | 3.30 | 3.92 | 3.94 | 3.91 |

In comparison to the pH data for Atrorosin-E of Table 1, the absorption spectrum of carminic acid is determined as above. Its absorption maxima are 495 nm, 525 nm, 565 nm. The pH stability data for carminic acid is shown below in Table 2:

TABLE 2

| Wavelength (nm) | pH 2.2 | pH 3.5 | pH 5 | pH 7.2 |
|---|---|---|---|---|
| 495 | 0.859 | 1.289 | 1.561 | 1.625 |
| 525 | 0.88 | 1.404 | 1.834 | 2.171 |
| 565 | 0.771 | 1.365 | 1.852 | 2.23 |

It is clear from the data shown in Tables 1 and 2 that compared with that of carminic acid, Atrorosin-E shows superior pH stability at low pH.

### Example 10: Temperature Stability of Atrorosin Compared to Betanin

100 mg of resulting powder from example 2, was dissolved in 250 ml in McIlvaine buffer pH 5. Buffer and atrorosin was mixed for 5 minutes before the solutions were subjected heating in an incubator at 60° C. and 80° C. to colorimetric analysis using spectrophotometer. The absorption maxima at 425 nm and 490 nm were determined in a microtiter plate with a 0.45 cm cell length with buffer as blank.

Absorption spectrum of Atrorosin-E is shown in table 3 after heating at 60° C. over a 24 hour period.

TABLE 3

| Wavelength | | | | | |
|---|---|---|---|---|---|
| (nm) | T0: Start | T1: 1 H | T2: 2 H | T3: 3 H | T4: 24 H |
| 425 | 2.03 | 2.01 | 1.98 | 1.78 | 0.78 |
| 490 | 3.55 | 3.53 | 3.42 | 3.11 | 1.31 |

In comparison to the data in Table 3, the absorption spectrum of betanin is determined as above. Its absorption maximum is at 530 nm. Table 4 shows heat stability at 60° C. over a 24 hour period.

TABLE 4

| Wavelength | | | | | |
|---|---|---|---|---|---|
| (nm) | T0: Start | T1: 1 H | T2: 2 H | T3: 3 H | T4: 24 H |
| 530 | 0.29 | 0.26 | 0.2 | 0.17 | 0.05 |

It is clear that betanin degrades significantly at 60° C. over a period of 24 hours, whereas atrorosin-E shows a much greater resistance towards heat mediated breakdown at this temperature and within the 24 hour span.

Absorption spectrum of Atrorosin-E is shown in table 5 after heating at 80° C. over a 3 hour period.

TABLE 5

| Wavelength | | | | | |
|---|---|---|---|---|---|
| (nm) | T0: Start | T1: 15 min | T2: 1 H | T3: 2 H | T4: 3 H |
| 425 | 2.04 | 2.00 | 1.66 | 1.23 | 0.99 |
| 490 | 3.48 | 3.38 | 2.86 | 2.13 | 1.71 |

In comparison to Table 5, the absorption spectrum of betanin is determined as above. Its absorption maximum is at 530 nm. Table 6 shows absorption spectrum of betanin after heating at 80° C. over a 3 hour period.

TABLE 6

| Wavelength | | | | | |
|---|---|---|---|---|---|
| (nm) | T0: Start | T1: 15 min | T2: 1 H | T3: 2 H | T4: 3 H |
| 530 | 0.28 | 0.19 | 0.07 | 0.05 | 0.03 |

It is clear that betanin degrades significantly at 80° C. over a period of 3 hours, whereas atrorosin-E shows a much greater resistance towards heat mediated breakdown at this temperature and within the 3 hour span.

### Example 11: Antioxidant Effect of Atrorosin

To assess antioxidant activity of Atrorosin-E, the common DPPH (2,2 Diphenyl-1-picrylhydrazyl) assay was used. A working solution of DPPH was made with a concentration of 220 μg/ml in ethanol. 1 g of the resulting powder from Example 2 was used to make a dilution row of atrorosin-E from 0 μM to 45 μM. 100 ml of each sample was reacted to 1000 μL of DPPH for 30 minutes in the dark before absorbance was measured.

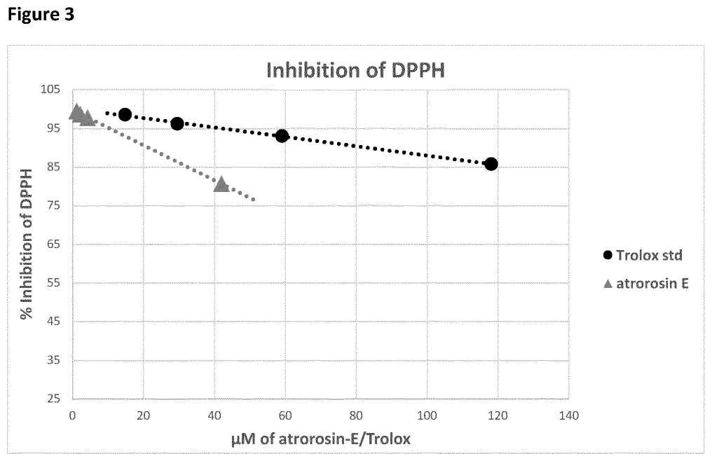

Antioxidant activity was determined by calculating the inhibition of emission of DPPH by measuring absorbance of DPPH at 517 nm before and after incubation with reactants. As Atrorosins have background emission at 517, the before sample was measured seconds after addition of atrorosins rather than before. Antioxidant activity of Atrorosin-E was compared to the standard Trolox (FIG. 3).

It is clear from the data presented in FIG. 3 that atrorosin-E is a far more potent antioxidant than the control antioxidant Trolox.

### Example 12: Genotoxicity of Atrorosin-E

Resulting powder from example 2 was sent to Cyprotox to asses for genotoxicity in a mini-Ames experiment. 43.2 mg of atrorosin was sent to Cyprotox. Mini-Ames test were conducted under the guidelines of OECD.

Table show data for Chrm-4=atrorosin and for Chrm-6=Carmine.

### AMES MPF: Individual Data.

Chrm-4

| | | | | | Assay Dec. 12, 2017 | | |
|---|---|---|---|---|---|---|---|
| TA 98 −S9 | | | | | | Fold | t-test |
| | | mean # | | | | increase | p-value |
| Conc. | | pos. | Corr. | | Base- | (over | (unpaired, 1- |
| (μg/ml] | n | Wells | mean | SD | line | baseline) | sided) |
| 0 | 12 | 0.67 | 1.00 | 0.65 | 1.32 | | |
| 62.5 | 3 | 0.00 | | 0.00 | | 0.00 | 0.0542 |
| 125 | 3 | 1.00 | | 0.00 | | 0.76 | 0.2022 |
| 250 | 3 | 2.00 | | 2.00 | | 1.52 | 0.0283 |
| 500 | 3 | 1.00 | | 1.73 | | 0.76 | 0.2892 |
| 1000 | 3 | 0.67 | | 0.58 | | 0.51 | 0.5000 |
| 2000 | 3 | 0.67 | | 1.15 | | 0.51 | 0.5000 |
| Pos. Contr | 3 | 42.00 | | 2.00 | | | |

Chrm-4

| | | | | | Assay Dec. 12, 2017 | | |
|---|---|---|---|---|---|---|---|
| TA 98 +S9 | | | | | | Fold | t-test |
| | | mean # | | | | increase | p-value |
| Conc. | | pos. | Corr. | | Base- | (over | (unpaired, 1- |
| (μg/ml] | n | Wells | mean | SD | line | baseline) | sided) |
| 0 | 12 | 1.33 | | 1.07 | 2.41 | | |
| 62.5 | 3 | 1.33 | | 1.15 | | 0.55 | 0.5000 |
| 125 | 3 | 0.67 | | 0.58 | | 0.28 | 0.1632 |
| 250 | 3 | 1.00 | | 1.00 | | 0.42 | 0.3175 |
| 500 | 3 | 0.67 | | 0.58 | | 0.28 | 0.1632 |
| 1000 | 3 | 1.67 | | 2.08 | | 0.69 | 0.3467 |
| 2000 | 3 | 0.00 | | 0.00 | | 0.00 | 0.0283 |
| Pos. Contr | 3 | 48.00 | | 0.00 | | | |

In comparison to the atrorosin tested above, similar 46.4 mg of carminic acid was also sent to Cyprotox to assess for genotoxicity.

Chrm-6

| | | | | | Assay Dec. 12, 2017 | | |
|---|---|---|---|---|---|---|---|
| TA 98 −S9 | | | | | | Fold | t-test |
| | | mean # | | | | increase | p-value |
| Conc. | | pos. | Corr. | | Base- | (over | (unpaired, 1- |
| (μg/ml] | n | Wells | mean | SD | line | baseline) | sided) |
| 0 | 12 | 0.67 | 1.00 | 0.65 | 1.32 | | |
| 62.5 | 3 | 0.33 | | 0.58 | | 0.25 | 0.2173 |
| 125 | 3 | 0.67 | | 0.58 | | 0.51 | 0.5000 |
| 250 | 3 | 0.33 | | 0.58 | | 0.25 | 0.2173 |
| 500 | 3 | 0.33 | | 0.58 | | 0.25 | 0.2173 |
| 1000 | 3 | 0.00 | | 0.00 | | 0.00 | 0.0542 |
| 2000 | 3 | 0.00 | | 0.00 | | 0.00 | 0.0542 |
| Pos. Contr | 3 | 38.00 | | 2.65 | | | |

Chrm-6

| | | | | | Assay Dec. 12, 2017 | | |
|---|---|---|---|---|---|---|---|
| TA 98 +S9 | | | | | | Fold | t-test |
| | | mean # | | | | increase | p-value |
| Conc. | | pos. | Corr. | | Base- | (over | (unpaired, 1- |
| (μg/ml] | n | Wells | mean | SD | line | baseline) | sided) |
| 0 | 12 | 1.33 | | 1.07 | 2.41 | | |
| 62.5 | 3 | 1.00 | | 1.00 | | 0.42 | 0.3175 |
| 125 | 3 | 0.67 | | 0.58 | | 0.28 | 0.1632 |
| 250 | 3 | 0.33 | | 0.58 | | 0.14 | 0.0750 |
| 500 | 3 | 0.00 | | 0.00 | | 0.00 | 0.0283 |
| 1000 | 3 | 0.00 | | 0.00 | | 0.00 | 0.0283 |
| 2000 | 3 | 0.33 | | 0.58 | | 0.14 | 0.0750 |
| Pos. Contr | 3 | 48.00 | | 0.00 | | | |

The mini-Ames study shows that both Atrorosin-E and carminic acid do not exert any genotoxic effects. This is a crucial requirement for a food colorant.

### Example 13: Pharmacokinetic Profile of Atrorosin

Resulting powder from example 2 was sent to Evotec to asses for the pharmacokinetic profile of atrorosin. 6 rats were used, 3 rats for IV injection of atrorosin and 3 rats for PO. The rats were injected or given through mouth gauge 2 mg/kg, and blood samples were taken at regular intervals.

| | | | | | | PO 2 mg/kg | | | | |
|---|---|---|---|---|---|---|---|---|---|---|
| | IV 2 mg/kg | | | | | | | | Mean/Median | |
| PK Parameter | 1 | 2 | 3 | Mean | SD | 4 | 5 | 6 | (Tmax) | SD |
| Dose (mg/kg) | 2 | 2 | 2 | 2 | — | 2 | 2 | 2 | 2 | — |
| Dose (μmol/kg) | 4 | 4 | 4 | 4 | — | 4 | 4 | 4 | 4 | — |
| C0/Cmax (ng/mL) | 9019 | 10685 | 10589 | 10098 | 935 | 4.35 | 5.35 | 3.68 | 4.46 | 0.84 |
| C0/Cmax (nM) | 16634 | 19708 | 19529 | 18623 | 1725 | 8.03 | 9.9 | 6.79 | 8.2 | 1.5 |
| Tmax (h) | — | — | — | — | — | 2 | 1 | 0.25 | 1 | — |
| t½ (h) | 1.3 | 0.8 | 1.9 | 1.3 | 0.6 | — | — | — | — | — |
| MRT (h) | 0.5 | 0.4 | 0.5 | 0.5 | 0.1 | — | — | — | — | — |
| Vdss (L/kg) | 0.5 | 0.5 | 0.5 | 0.5 | 0.05 | — | — | — | — | — |
| CL/CL_F (mL/min/kg) | 14 | 18 | 16 | 16 | 2 | — | — | — | — | — |
| AUCinf (ng · hr/mL) | 2336 | 1822 | 2063 | 2073 | 257 | — | — | — | — | — |
| AUCinf (nM · hr) | 4308 | 3360 | 3804 | 3824 | 474 | — | — | — | — | — |
| AUC0-t (ng · hr/mL) | 2330 | 1818 | 2055 | 2067 | 256 | 6.35 | 7.6 | 5.76 | 6.6 | 0.9 |
| AUC0-t (nM · hr) | 4297 | 3352 | 3790 | 3813 | 473 | 11.7 | 13.9 | 10.6 | 12.1 | 1.7 |
| Clast (ng/mL) | 3.26 | 3.45 | 2.73 | 3.15 | 0.375 | 4.35 | 2.6 | 2.88 | 3.28 | 0.94 |
| Bioavailability (%) | — | — | — | — | — | — | — | — | — | — |
| Using AUCinf | | | | | | | | | | |
| Bioavailability (%) | — | — | — | — | — | 0.3% | 0.4% | 0.3% | 0.3% | 0.0% |
| Using AUC0-t | | | | | | | | | | |
| Number of Points | 3 | 4 | 3 | — | — | — | — | — | — | — |
| used for Lambda z | | | | | | | | | | |
| AUC % Extrapolation | 0.3 | 0.2 | 0.4 | — | — | — | — | — | — | — |
| to infinity | | | | | | | | | | |
| Tlast (h) | 8 | 6 | 8 | — | — | 2 | 2 | 2 | — | — |

In comparison to example 10, similar carminic acid was also sent to Evotec to assess the pharmacokinetic profile.

| | | | | | | PO 3 mg/kg | | | | |
|---|---|---|---|---|---|---|---|---|---|---|
| | IV 1 mg/kg | | | | | | | | Mean/Median | |
| PK Parameter | 1 | 2 | 3 | Mean | SD | 4 | 5 | 6 | (Tmax) | SD |
| Dose (mg/kg) | 1 | 1 | 1 | 1 | — | 3 | 3 | 3 | 3 | — |
| Dose (μmol/kg) | 2.0 | 2.0 | 2.0 | 2.0 | — | 6.1 | 6.1 | 6.1 | 6.1 | — |
| C0/Cmax (ng/mL) | 7095 | 6542 | 11003 | 8213 | 2432 | — | — | — | — | — |
| C0/Cmax (nM) | 14410 | 13286 | 22346 | 16681 | 4939 | — | — | — | — | — |
| Tmax (h) | — | — | — | — | — | — | — | — | — | — |
| t½ (h) | 3.7 | 3.4 | 3.4 | 3.5 | 0.2 | — | — | — | — | — |
| MRT (h) | 3.2 | 3.3 | 3.9 | 3.5 | 0.3 | — | — | — | — | — |
| Vdss (L/kg) | 0.3 | 0.3 | 0.3 | 0.3 | 0.0 | — | — | — | — | — |
| CL/CL_F (mL/min/kg) | 1.4 | 1.3 | 1.2 | 1.3 | 0.1 | — | — | — | — | — |
| AUCinf (ng · hr/mL) | 11497 | 12597 | 13408 | 12501 | 959 | — | — | — | — | — |
| AUCinf (nM · hr) | 23350 | 25583 | 27230 | 25388 | 1948 | — | — | — | — | — |
| AUC0-t (ng · hr/mL) | 11431 | 12535 | 13332 | 12433 | 954 | — | — | — | — | — |
| AUC0-t (nM · hr) | 23216 | 25457 | 27076 | 25250 | 1938 | — | — | — | — | — |
| Fraction Absorbed | — | — | — | — | — | — | — | — | — | — |
| Clast (ng/mL) | 12.3 | 12.7 | 15.3 | 13.4 | 1.6 | — | — | — | — | — |
| Bioavailability (%) | — | — | — | — | — | — | — | — | — | — |
| Using AUCinf | | | | | | | | | | |
| Bioavailability (%) | — | — | — | — | — | — | — | — | — | — |
| Using AUC0-t | | | | | | | | | | |
| Number of Points | 3 | 4 | 3 | — | — | — | — | — | — | — |
| used for Lambda z | | | | | | | | | | |
| AUC % Extrapolation | 0.6 | 0.5 | 0.6 | — | — | — | — | — | — | — |
| to infinity | | | | | | | | | | |
| AUC % Back | 4.7 | 4.2 | 5.1 | — | — | — | — | — | — | — |
| Extrapolation to C0 | | | | | | | | | | |
| Tlast (h) | 24 | 24 | 24 | — | — | — | — | — | — | — |

These results indicate that atrorosin is safe when ingested, it has a bioavailability of 0.3% whereas for carminic acid it could not be determined. Atrorosin has a moderate plasma clearance of 16±2 mL/min/kg where as carminic acid had a low plasma clearance of 1.3±0.1 mL/min/kg. The half-life of atrorosin is 1.3±0.6 h whereas for carminic acid it was estimated to be 3.5±0.2 h.

### Example 14: Use of Atrorosin Dye for Binding to Collagen

Collagen is the typical material used to make sausage casings. The harsh conditions of manufacturing do not allow producers to use betanin, so they either use Red #3, a synthetic colorant, or carmine. However, due to the poor consumer reputation of carmine it is industrially relevant to replace carmine with a different natural color solution.

Atrorosin dye was tested for its ability to survive a mimic of the harsh conditions that collagen casing undergoes in the manufacturing process.

1 g of collagen was dissolved in 50 mL of 0.1 M acetic acid and stirred for 20 minutes at room temperature.

Step 0: A casing matrix was prepared by mixing 10% collagen mix, with 45% glycerol (99%) and 45% of demineralized water. The pH of the casing matrix was adjusted to pH 1.4 with 5M HCl to make the conditions extreme.

Step 1: To 100 ml of casing matrix, 15 mg of resulting powder from Example 2 was added to the matrix and stirred for 10 minutes.

Step 2: After 10 minute incubation time, the pH of the matrix was adjusted to pH 10 with 2M NaOH.

Step 3: After 45 minute incubation period, the pH of the matrix was seen to be steady at pH 10, and was lowered to pH 5 with 5 M HCl.

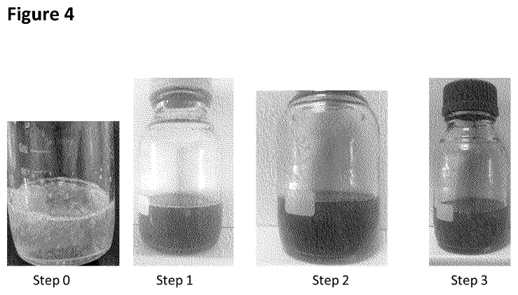

As can be seen from the FIG. 4, the casing matrix retained its color throughout the prolonged incubation at harsh conditions of high and low pH, and the subsequent changes in pH.

### Example 15: Use of Atrorosin Dye for Coloring of Vegan Burger Patty

For vegan burger patty only a few options are available to color the patty a nice red shade. Carminic acid/carmine cannot be used as it is not vegan, so the majority uses either betanin from beetroot at a high dose (1%) or anthocyanins (1%) but these options are unsatisfactory in getting a nice red shade both before and after frying of the patty.

In this example, evidence of the high stability of the atrorosins of the invention is shown, by adding to a vegan burger patty, and colouring is measured prior to and after frying. Results are shown in the below table.

The CIEL*a*b* color system to measure the lightness (L*), red and green (a*) (negative indicate green, while positive indicate red), yellow and blue (b*) (negative indicate blue and positive indicate yellow).

### Burger Patty Base:

Water: 43%

Textured vegetable protein: 27%

Coconut oil: 5%

Rapeseed oil: 13%

Potato flour: 10%

Methylcellulose: 2%

Atrorosin dye was added in concentrations of 87.5 PPM and 130 PPM.

| Burger | Atrorosin-E (PPM) | L | A | B |
|---|---|---|---|---|
| Burger 1 Pre-frying | 87.5 | 57.67 | 17.93 | 32.92 |
| Burger 1 Post-frying | 87.5 | 37.96 | 20.74 | 24.92 |
| Burger 2 Pre-frying | 130 | 56.22 | 23.26 | 16.39 |
| Burger 2 Post-frying | 130 | 43.49 | 21.93 | 21.69 |

wax. When the base ingredients had melted, 0.5 gram of Atrorosin-Lake was added to the base.

### Example 16: Use of Atrorosin Lake for Lipstick

Red is the most used color for lipsticks and cosmetic products. In the cosmetic industry, the many shades of red are typically created by mixing a lot of colors, and also here the search for new natural colorants is ongoing to be able to launch new exciting colors. Atrorosin-E lake as made from example 8 was in this example used to color a simple lipstick.

5 grams of beeswax was slowly heated, and 2 grams of coconut oil was added together with 2.5 grams of carnaboa wax. When the base ingredients had melted, 0.5 gram of Atrorosin-Lake was added to the base. The lipstick base was stirred for 5 minutes and poured into a suitable container for drying at room temperature. After 2 hours of drying the lipstick was ready for use.

As reference Atrorosin dye was tested, however it was not easily dissolved into the base, and the final lipstick had uneven coloring.

Shelf-life testing by allowing the two products to be stored at room temperature for 4 weeks also showed that the Atrorosin-Lake could keep its color without visible tinctorial decay, whereas the Atrorosin dye lipstick was almost completely faded after 4 weeks. This experiment shows the stability at room temperature of the atrorosin lakes of the invention, and the potential of using these for cosmetics, i.e. for lipsticks.

### Example 17: Use of Atrorosin Lake for Coloring of Beverages

Beverages and especially soft drinks it is common to have red shades. The acidic environment of soft drinks makes many colors unsuitable. In this example atrorosin is used to color beverages.

The CIEL*a*b* color system to measure the lightness (L*), red and green (a*) (negative indicate green, while positive indicate red), yellow and blue (b*) (negative indicate blue and positive indicate yellow).

### Model Beverage Medium Concentrate:

Sucrose: 43%

Potassium Sorbate: 0.09%

Sodium Benzoate 0.07%

Citric acid 0.86%

MiliQ water: 55.98%

pH of the concentrate is 7.3

Soft drinks were prepared by adding 65 ml of beverage concentrate to 185 ml of carbonated water and lower the pH to 3 with citric acid. Atrorosin-E was added to be to make different colors of red.

| Beverage | Atrorosin-E (PPM) | L | A | B |
|---|---|---|---|---|
| Beverage 1 | 26.25 | 40.92 | 45.66 | 33.92 |
| Beverage 2 | 39.4 | 36.55 | 49.61 | 44.37 |
| Beverage 3 | 70 | 29.5 | 52.76 | 50.77 |

### Example 17: Use of Atrorosin Lake for Coloring of Beverages

Beverages and especially soft drinks it is common to have red shades. The acidic environment of soft drinks makes many colors unsuitable. In this example atrorosin is used to color beverages.

The CIEL*a*b* color system to measure the lightness (L*), red and green (a*) (negative indicate green, while positive indicate red), yellow and blue (b*) (negative indicate blue and positive indicate yellow).

### Model Beverage Medium Concentrate:

Sucrose: 43%

Potassium Sorbate: 0.09%

Sodium Benzoate 0.07%

Citric acid 0.86%

MiliQ water: 55.98%

pH of the concentrate is 7.3

Soft drinks were prepared by adding 65 ml of beverage concentrate to 185 ml of carbonated water and lower the pH to 3 with citric acid. Atrorosin-E was added to be to make different colors of red.

| Beverage | Atrorosin-E (PPM) | L | A | B |
|---|---|---|---|---|
| Beverage 1 | 26.25 | 40.92 | 45.66 | 33.92 |
| Beverage 2 | 39.4 | 36.55 | 49.61 | 44.37 |
| Beverage 3 | 70 | 29.5 | 52.76 | 50.77 |

### Example 18: Use of Atrorosin for Coloring of Candy

Candies and confectionary are typically colored red. In this example, we have tested atrorosins as a coloring agent for hard and soft candy.

The CIEL*a*b* color system to measure the lightness (L*), red and green (a*) (negative indicate green, while positive indicate red), yellow and blue (b*) (negative indicate blue and positive indicate yellow.

### Hard Candy Base Recipe:

| Ingredient | Weight |
|---|---|
| Water | 173 g |
| Glucose syrup | 245 g |
| White sugar | 400 g |

### Soft Candy Base Recipe:

| Ingredient | Weight |
|---|---|
| Water | 170 g |
| Glucose syrup | 225 g |
| White sugar | 225 g |
| Gelatin | 44 g |

Hard candy was prepared by mixing the ingredients of the base recipe, and heating it slowly to 127 C. At this temperature, Atrorosin-E at varying concentrations was added to the desired coloration effect. The colored sugar mixture continued to heat until 148° C. The candy syrup was then poured into molds and cooled at room temperature.

Soft candy was prepared by creating a sugar mixture from glucose syrup and sugar and slowly heating it to 100 C. At this temperature, Atrorosin-E at varying concentrations was added to the desired effect and stirred. In separate bowl, a gelatin mixture was prepared by combining gelatin and cold water.

The sugar mixture was combined with the gelatin mixture to create a soft candy. The soft candy was poured into molds and cooled at 4 C for 24 hours until set.

Atrorosin-E was added to the candy bases in different concentrations to give different color shades.

| Candy | Atrorosin-E (PPM) | L | A | B |
|---|---|---|---|---|
| Hard candy 1 | 43.75 | 56.32 | 39.83 | 31.99 |
| Hard candy 2 | 87.5 | 39.82 | 51.19 | 34.42 |
| Soft candy 1 | 8.75 | 51.88 | 21.22 | 6.14 |
| Soft candy 2 | 87.5 | 25.95 | 15.92 | 4.69 |

From the data in the above table, it is clear that the atrorosins of the invention are useful in preparing both soft and hard candy, i.e. that the atrorosins are able to withstand the harsh heating conditions during the manufacture of candy.

### REFERENCES

1. Dufossé, L. Microbial production of food grade pigments. Food Technol. Biotechnol. 44, 313-321 (2006).
2. Scotter, M. J. Emerging and persistent issues with artificial food colours: natural colour additives as alternatives to synthetic colours in food and drink. Qual. Assur. Saf. Crop. Foods 3, 28-39 (2011).
3. Amchova, P., Kotolova, H. & Ruda-Kucerova, J. Health safety issues of synthetic food colorants. Regul. Toxicol. Pharmacol. 73, 914-922 (2015).
4. Wissgott, U. & Bortlik, K. Prospects for new natural food colorants. Trends Food Sci. Technol. 7, 298-302 (1996).
5. Esquivel, P. Betalains. Handb. Nat. Pigment. Food Beverages Ind. Appl. Improv. Food Color 81-99 (2016) doi:10.1016/B978-0-08-100371-8.00004-X.
6. von ELBE, J. H., MAING, I. -Y & AMUNDSON, C. H. Color Stability of Betanin. J. Food Sci. 39, 334-337 (1974).
7. Garin, T. A. & Vogel, G. J. Process for concentrating and isolating a beet colorant. (1983).
8. Dapson, R. W. The history, chemistry and modes of action of carmine and related dyes.

Biotech. Histochem. 82, 173-187 (2007).
9. Müller-Maatsch, J. & Gras, C. The ‘Carmine Problem’ and Potential Alternatives. Handbook on Natural Pigments in Food and Beverages: Industrial Applications for Improving Food Color vol. 80 (Elsevier Ltd, 2016).

1. A method for preparing an Atrorosin food coloring composition from a fermentation broth, comprising:
a. Removing the biomass and other macro-sized constituents by membrane filtration;
b. Removing proteins, peptides and other constituents by ultrafiltration through an ultrafiltration membrane with a cutoff between 1 kDa to 100 kDa;
c. Performing acid precipitation of the Atrorosin of the permeate of step b, wherein the pH is lowered to be below the pKa of the Atrorosin;
d. Filtering the precipitate of step c by membrane filtration (with a membrane having a pore size below 1 μm;
e. Adding a buffer to the precipitate of step d to increase the pH to above the pKa of the Atrorosin of the precipitate; and
f. Drying the mixture of step e to remove water to make an Atrorosin powder comprising a salt composition of Atrorosin and salt of the buffer.

2-32. (canceled)

33. The method according to claim 1, wherein the filter of step a has a pore size of between 1 μm and 40 μm.

34. The method according to claim 1, wherein the Atrorosin is Atrorosin-E, and the precipitation in step c is performed by lowering the pH of the permeate to a pH lower than pH 1.7.

35. The method according to claim 33, wherein the pH is lowered by addition of anyone of the acids selected from the list of: Hydroiodic (HI), Hydrobromic (HBr), Perchloric (HClO4), Hydrochloric (HCl), Chloric (HClO3), Sulphuric (1) (H2SO4), Nitric (HNO3), Hydronium ion (H3O+), Iodic (HlO3), Oxalic (1) (H2C2O4), Sulphurous (1) (H2SO3), Sulphuric (2) (HSO4-), Chlorous (HClO2), or Phosphoric (1) (H3PO4).

36. The method according to claim 35, wherein the acid is selected from hydrochloric acid, nitric acid, phosphoric acid or sulphuric acid.

37. The method according to claim 1, wherein the buffer of step e is citrate buffer.

38. The method according to claim 1, wherein the drying is performed by lyophilisation, spray drying, or evaporation.

39. An Atrorosin salt produced by the method according to steps a-d of claim 1.

40. A powder obtained from step f of the method according to claim 1.

41. The method according to claim 1, wherein the Atrorosin is Atrorosin-E.

42. A composition comprising Atrorosin, wherein at least 80% of the Atrorosin is cis Atrorosin, and less than 20% is trans Atrorosin.

43. The composition according to claim 42, wherein the Atrorosin is Atrorosin-E, and less than 20% by weight is trans Atrorosin-E.

44. The composition according to claim 42, wherein less than 15% by weight is trans Atrorosin.

45. The composition according to claim 42, wherein less than 10% by weight is proteins, peptides or carbohydrates.

46. A method of producing a lake comprising providing the Atrorosin food coloring composition prepared by claim 1 in a method performed to produce a lake.

47. The method according to claim 46, wherein the lake is made by coupling of the Atrorosin to a metal.

48. The method according to claim 47, wherein the Atrorosin is coupled to anyone of aluminium, calcium, barium, zinc, sodium or copper.

49. A dye comprising the Atrorosin food coloring composition prepared by claim 1.

50. The dye according to claim 49, wherein the Atrorosin is formulated with sugars, or other carbohydrates.

51. The dye according to claim 50, wherein the carbohydrate is selected from maltodextrin, cyclodextrin or pectin.

52. A method of preparing a food, non-food product, or cosmetic comprising incorporating the Atrorosin food coloring composition prepared by claim 1 into a food, non-food product, or cosmetic.

53. A product comprising the Atrorosin food coloring composition prepared by claim 1, wherein the product is selected from a food, a non-food product or a cosmetic.

54. A kit for coloring or preserving a product, wherein the kit comprises at least one Atrorosin food coloring composition prepared by claim 1, wherein the Atrorosin food coloring composition is supplied in a container, and wherein the product is selected from a food, a non-food or a cosmetic.

55. The kit according to claim 54, wherein the kit further comprises an additional pigment, antioxidant or preservative.

56. The method according to claim 52, wherein the food is a dairy product.

57. The method according to claim 52, wherein the product is a food that is mixed with oil or fat.

58. The method according to claim 52, wherein the product is a food having a pH below 7.

59. The method according to claim 52, wherein the product is a food, which after addition of the Atrorosin will be heated.

60. The method according to claim 59, wherein the food will be heated to at least 50 degrees C.

61. The method according to claim 59, wherein the food product is a meat substitute.

62. The method according to claim 52, wherein the product is a beverage.

63. The method according to claim 52, wherein the product is acidic and will be heated.
